(19) United States
(12) Patent Application Publication
(10) Pub. No.: US 2017/0303848 A1
(43) Pub. Date: Oct. 26, 2017

(54) Infravein Vasculature Visualization Device

(71) Applicants: Kreg Zimmern, San Antonio, TX (US); Kristen Hamalainen, Aransas Pass, TX (US); Andrew Shiels, San Antonio, TX (US); Sanjiv Patel, Georgetown, TX (US); Teja Guda, San Antonio, TX (US)

(72) Inventors: Kreg Zimmern, San Antonio, TX (US); Kristen Hamalainen, Aransas Pass, TX (US); Andrew Shiels, San Antonio, TX (US); Sanjiv Patel, Georgetown, TX (US); Teja Guda, San Antonio, TX (US)

(73) Assignee: THE BOARD OF REGENTS OF THE UNIVERSITY OF TEXAS SYSTEM, Austin, TX (US)

(21) Appl. No.: 15/498,183

(22) Filed: Apr. 26, 2017

Related U.S. Application Data
(60) Provisional application No. 62/327,818, filed on Apr. 26, 2016.

(51) Int. Cl. A61B 5/00; A61M 5/42; G09B 19/00; G09B 5/00

(52) U.S. Cl. CPC A61B 5/489; G09B 19/00; G09B 5/00; A61B 5/0077; A61M 5/427; A61B 2562/185

(57) ABSTRACT

Embodiments of the devices described herein are directed to a vein imaging system which adds user protection, variable positioning, and portability to an established imaging modality, near infrared (NIR) videography and videogrammetry.

This application claims priority to U.S. Application No. 62/327,818 filed Apr. 26, 2016, which is incorporated herein by reference in its entirety.

### BACKGROUND

There are various other approaches to venous tissue visualization. Most use the same basic principal of near infrared (NIR) spectrum absorbance, but the delivery of the information varies. There are two main methods of delivery, by projecting and image onto the skin and by using a separate display like a computer or television screen. Projection of the image is the modality for devices like the Vein Viewer from ChristieMed and AccuVein. The draw backs for these products is they are not typically hands free, unless they are mounted on bulky apparatus. The resolution of the projection is very sensitive to the working distance of the device and the ambient light in the environment. They also must typically be plugged in to a wall outlet, and when they do have batteries they are large and heavy. This makes them impractical for use outside the clinical setting.

The display modality devices are exemplified by the Evena Eyes-on, VueTek, and the Portable Vein finder from Wuxi Biomedical Technology. The Evena and VueTek product are both head mounted and fixed position displays, restricting the field of view. The Wuxi Product is a desk top device that must stay plugged into the wall, and have room near the patient for the base of the product. This limits its ability to be used in close quarters like those of a mobile blood center or for use outside the clinical setting. In addition, The Evena and VueTek products have glass displays and are very fragile, and cannot be reduced in size from their deployed form factor.

### SUMMARY

Various aspects of the device described herein solves many of the aforementioned problems associated with the existing products. In certain embodiments the device is hands and cord free, a completely modular device that is small enough to be easily packed in a travel bag or medical kit for transportation. It is designed for rugged use and to withstand abuse from use in an emergency or battlefield situation. It is a display type device, but one that offers a nearly unobstructed field of vision and situational awareness. As such it maintains its functionality in all bright or dark ambient conditions. In certain aspects the device can be configured to provide protection for the patient and/or user against contamination or contact with a pathogen, for instance a device can include a splash guard to protect a user against blood borne pathogens from the patient. Embodiments of the device will also not be limited to a head mount modality, but will also be able to be quickly and securely mounted to a variety of surfaces, including mobile blood donor chairs. The display will not be at a fixed distance from the user's eyes, as it is in the Evena and VueTek, but be completely adjustable to the user's comfort, while maintaining accuracy and sensitivity.

Near infrared vein imaging systems allow the operator to identify veins based on their absorbance behavior in the presence of near infrared light. Deoxyhemoglobin in the veins absorbs near infrared light while surrounding tissues, e.g., fat, skin, etc., scatter and reflect near infrared light.

The device adds user protection by attaching a blood spatter shield, thereby protecting the clinician and surrounding personnel from exposure to potentially infected blood during venipuncture. The splatter shield will be easily cleanable and sterilizable without compromising transparency. The splatter shield will be designed to meet OSHA requirements for personal protective equipment for users who handle blood.

The device adds portability by providing the user the ability to mount the device to a variety of surfaces in many environments, including mobile ambulances, trauma centers, mobile and non-mobile blood donation stations, and headgear worn by the user. This will be realized by developing a modular, central unit and environment-specific mounts.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of one embodiment of a battery housing with attachment mechanism.

FIG. 2. Illustration of one embodiment of a dual camera assembly including a dual camera, flexible support, and a battery/attachment housing.

FIG. 3. Illustration of one embodiment of a dual camera assembly and a battery/attachment housing.

FIG. 4. Illustration of one embodiment of a head mountable assembly.

FIG. 5. Illustration of the components of one embodiment of a head mountable assembly.

FIG. 6. Illustration of the components of one embodiment of a dual camera assembly.

FIG. 7. Illustration of the components of one embodiment of a battery/attachment assembly.

FIG. 8. Illustration of a support platform.

FIG. 9. Illustration of a mounting bracket attached to a shield.

FIG. 10. Illustration of a dual camera assembly attached to a shield.

FIGS. 11A-11B. Illustration of head mountable assembly with flexible support. (FIG. 11A) frontal view. (FIG. 11B) top view.

FIGS. 12A-12B. Illustration of one embodiment of a dual camera assembly attached to a gooseneck support. (FIG. 12A) frontal view with cameras. (FIG. 12B) back view with display.

FIGS. 13A-13C. Illustration of one embodiment of a dual camera assembly. (FIG. 13A) frontal view. (FIG. 13B) back view. (FIG. 13C) top perspective view.

FIGS. 14A-14C. Illustration of one embodiment of a dual camera assembly attached to a gooseneck support with a modular attachment base. (FIG. 14A) left frontal perspective view. (FIG. 14B) right frontal perspective view. (FIG. 14C) back view.

FIG. 15. Illustration of one embodiment of a protective shield, front right perspective view.

FIGS. 16A-16C. Illustration of one embodiment of a dual camera and protective shield assembly attached to a gooseneck support with a modular attachment base. (FIG. 16A) top right view. (FIG. 16B) right perspective view. (FIG. 16C) back perspective view with transparent cube to demonstrate the three-dimensional aspect of the protective shield.

### DESCRIPTION

Certain embodiments of the present invention are called Infravein vasculature visualization devices. In certain aspects the device is intended to facilitate peripheral vein visualization. In one embodiment of the device a housing is intended to be mounted on a surface or support, e.g., blood donor chair, gurney, wall, or a table. In certain aspects the housing contains two independent cameras and a built in or removable splatter shield to offer protection against blood borne pathogens. In certain aspects the cameras are positioned parallel or at an approximately 90, 80, 70, 60, 50, 45 to 30, 20, 10 degree angle relative to each other. The second device design is a small housing design that can be head or head gear mountable. The head mountable device can be easily stored and transported by first responders or battlefield medics and attached to various surface or supports. In certain aspects the support is a head gear, helmet, or harness attached to a body. Both devices use the same core NIR visualization technology, are hands free and cordless, and offer different advantages to different users with different requirements.

The devices described herein are used to aid routine and non-routine venipuncture in many settings. Problems which arise in these settings pertain to the difficulty that clinicians, or healthcare personnel encounter when attempting to visually identify, palpate, and access peripheral veins. These include not only mundane variations such as pigmentation, individual vein depth, skin tissue composition, and individual blood pressure, but also variations associated with comorbid medical conditions, including profound burns, scar tissue, and deflated vasculature. In addition to the clinical practice applications, the devices described herein can be used for clinical training. For example the device can be utilized in conjunction with existing phlebotomy training techniques to hasten venipuncture proficiency in both normal and complicated conditions.

The device described herein is a vein imaging system which adds user protection, variable positioning, and portability to an established imaging modality, near infrared (NIR) videography and videogrammetry. Videogrammetry is a measurement technology in which the three-dimensional coordinates of points on an object are determined by measurements made in two or more video images taken from different angles. Images can be obtained from two cameras which simultaneously view the object or from successive images captured by the same camera with a view of the object. The device design allows the camera, light source, and display component to be configured and manufactured to be durable and damage resistant The durability facilitates operation in both clinical and extreme environments.

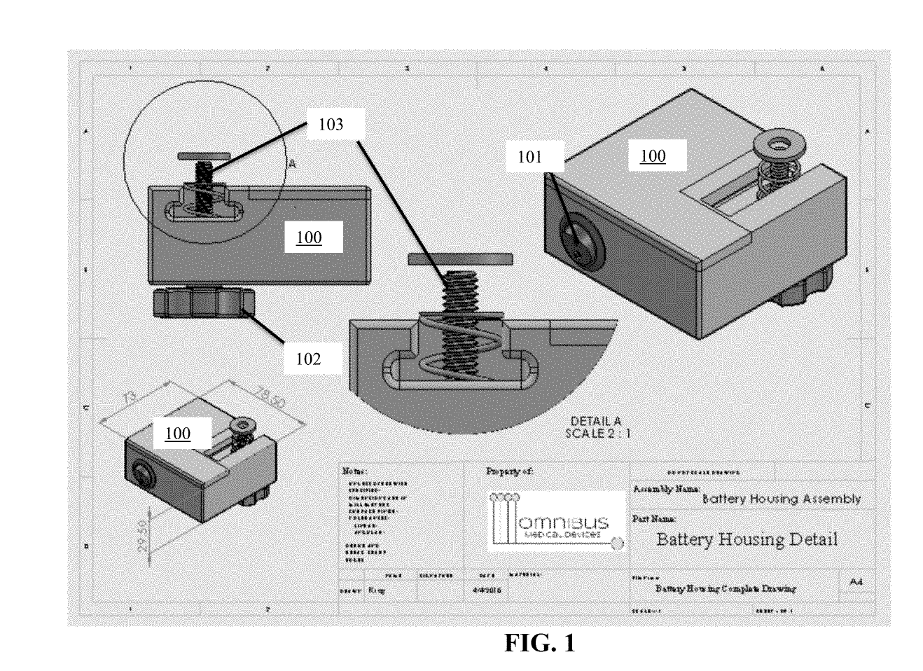

The battery housing/attachment base 100 is illustrated in more detail in FIG. 1. The battery housing is configured with power switch 101 and an attachment screw 103. Attachment screw 103 can be secured to a support by adjustment knob 102.

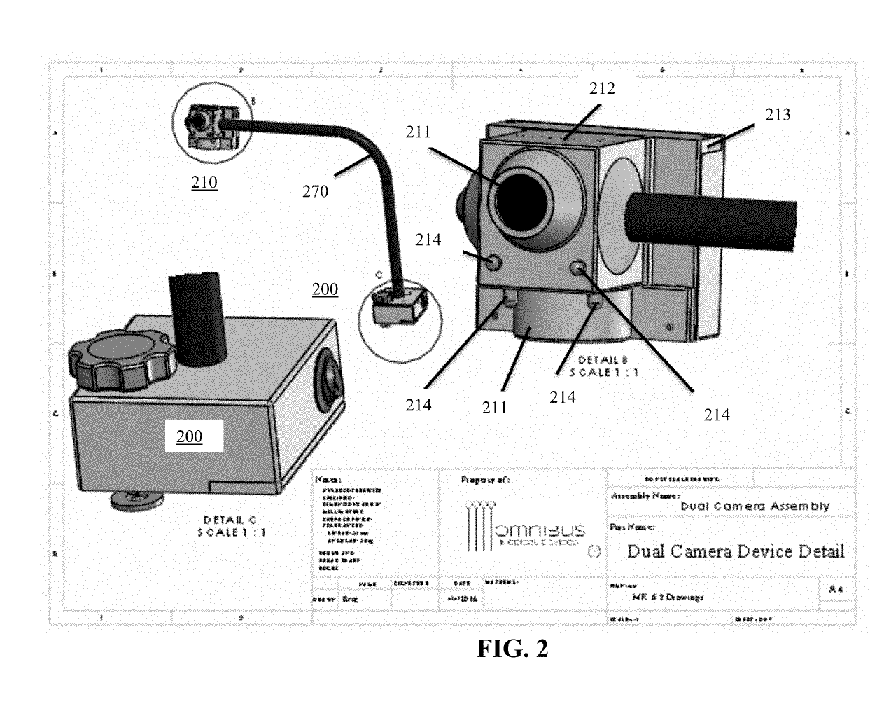

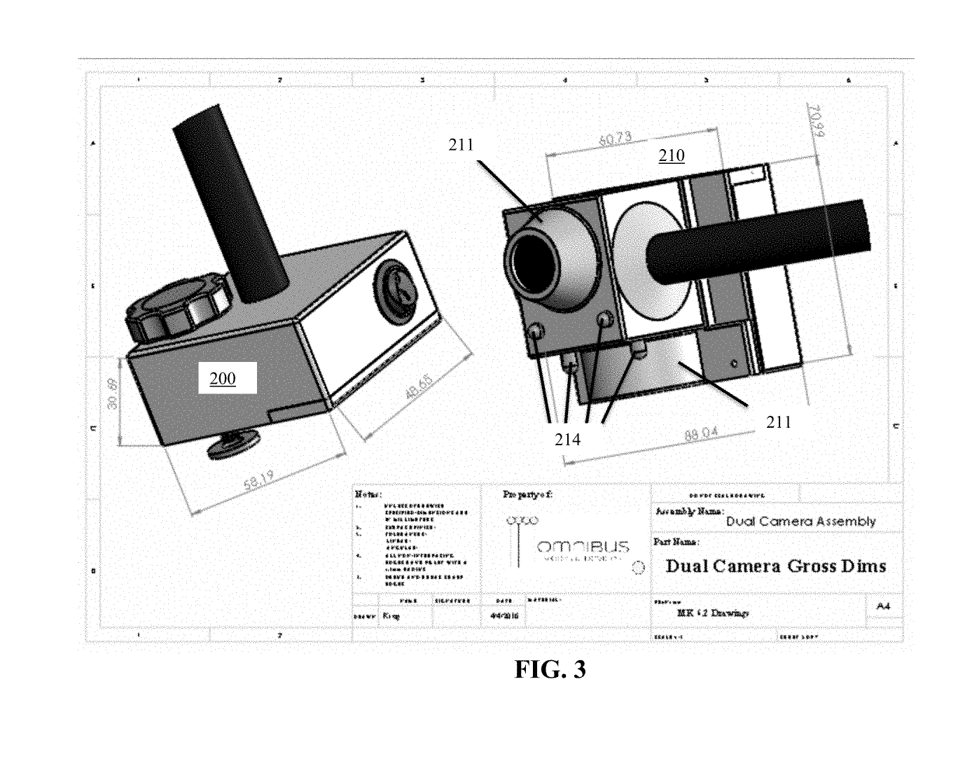

FIG. 2 and FIG. 3 illustrates one embodiment of a device that includes a battery housing/attachment base coupled to a camera assembly by a flexible support 215. FIG. 2 depicts battery housing/attachment base 200 flexibly coupled to camera assembly 210. The camera assembly comprises a lens assembly 211 including an optical filter. Lens assembly being configured to be used in conjunction with near infrared (NIR) LED 214. The lens and other components being assembled in housing 212 and housing insert 213.

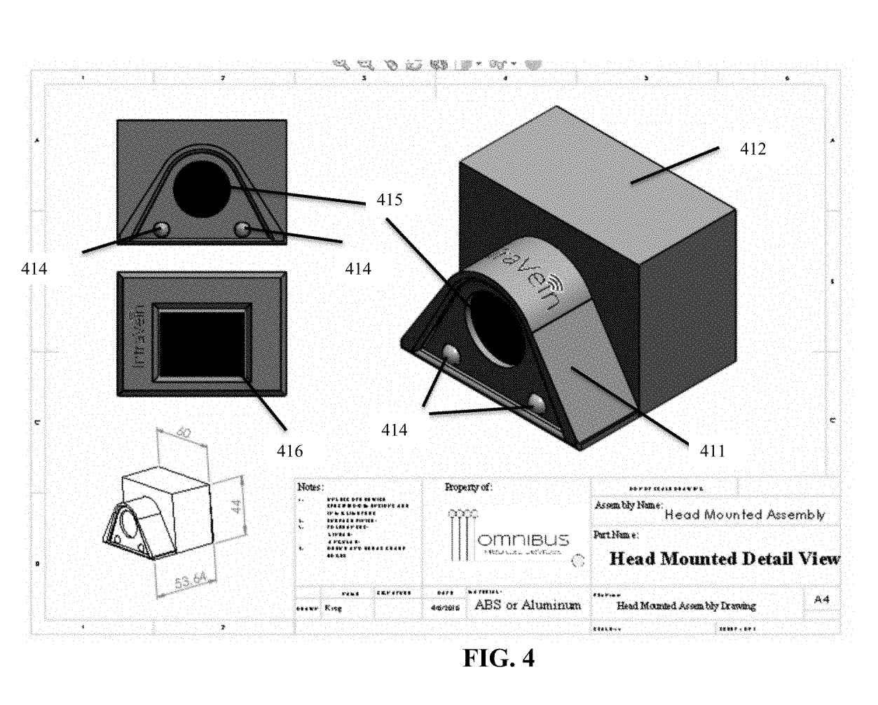

FIG. 4 illustrates a head mounted embodiment of a device of the present invention. FIG. 4 shows lens assembly 411 in housing 412. The lens assembly including optical filter 415 and NIR LED 414. Display 416 is assemble on the back of housing 412.

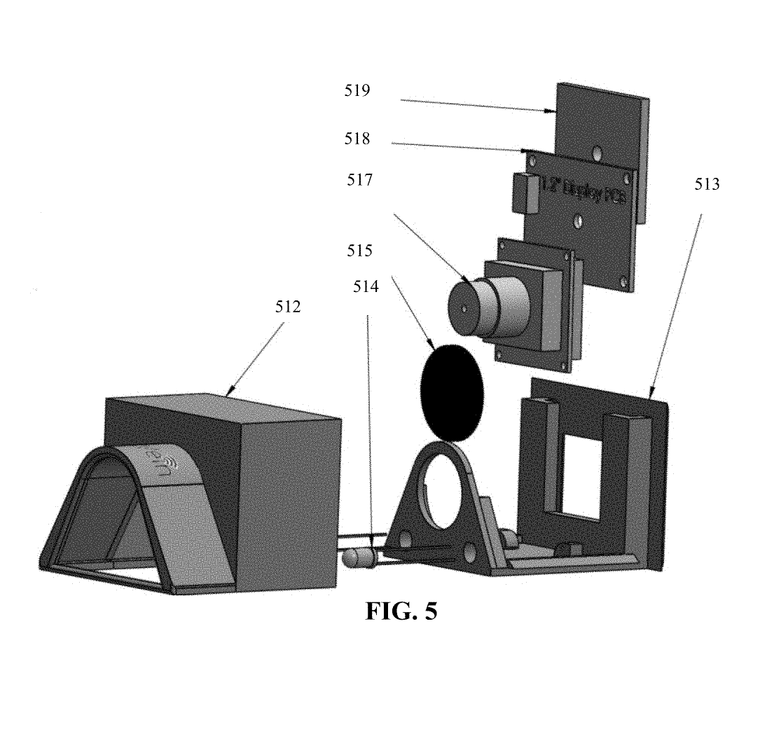

FIG. 5 illustrates an exploded view of a mountable camera assembly. Shown in FIG. 5 is housing cover 512 containing housing insert 513. Assembled with housing insert 513 are a light source including NIR LED 514 and camera 517. Camera 517 is assembled with optical filter 515 and display components 518 and 519.

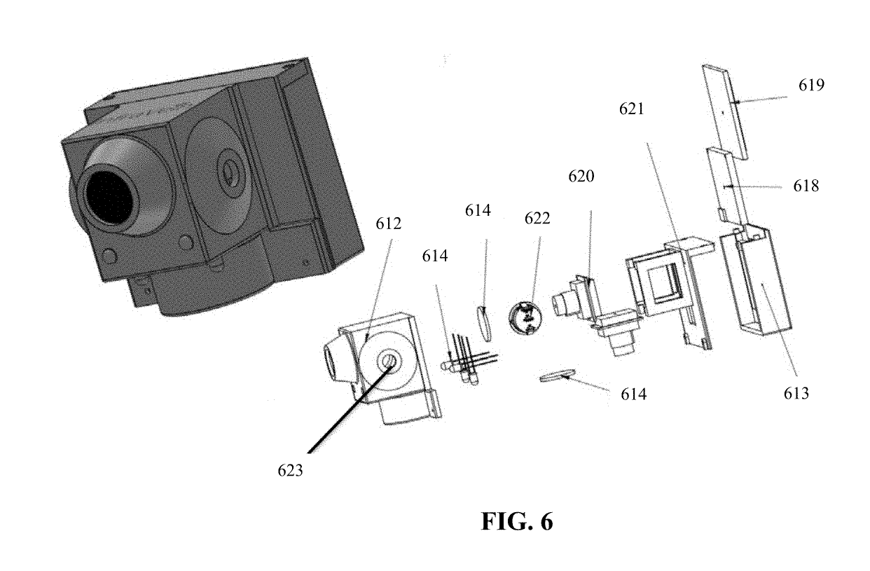

FIG. 6 illustrates one embodiment of a dual camera assembly, see also FIG. 2 and FIG. 3. Housing cover 612 is assemble with housing insert 613. Housing insert 613 is assembled with dual camera insert 621. Dual camera insert 621 is further assemble with cameras 620. Light sources 614 are also included in the assembly as well as optical filter 614 and display components 618 and 619. Rocker switch 622 is configured to power on and power off the camera assembly. Also illustrated are attachment sites 623 that are configured to receive a support arm or the like.

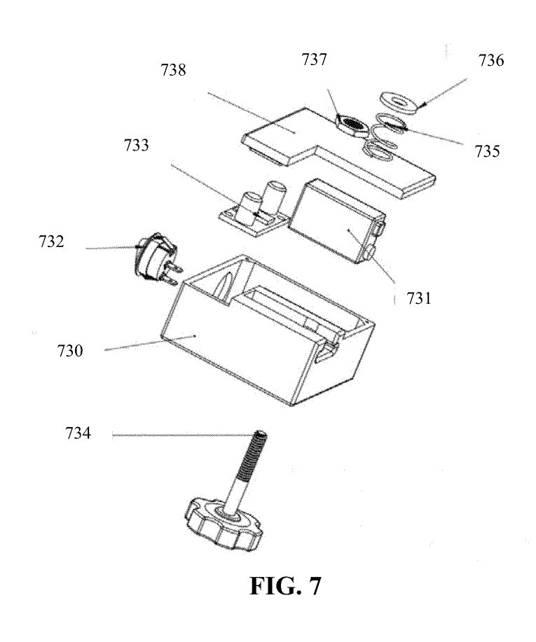

FIG. 7 illustrates one embodiment of a battery/attachment base. Battery housing 730 and battery housing cap 738 are assembled with battery 731 which is connected to switch 732 and voltage regulator 733. The Battery housing is configured to provide for attachment via screw 734 in combination with spring 735, washer 736 and nut 737 accessories.

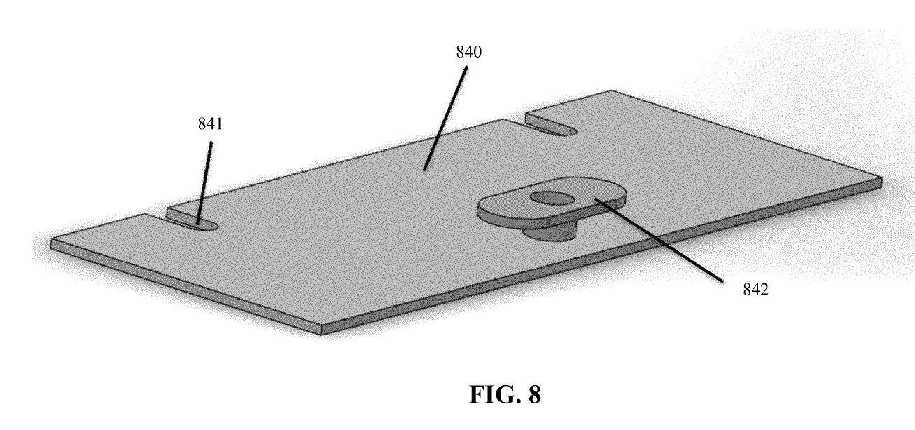

FIG. 8 illustrates a support platform or attachment plate 840 having notches 841 and a T-bar with hole 842 configured to receive a bolt.

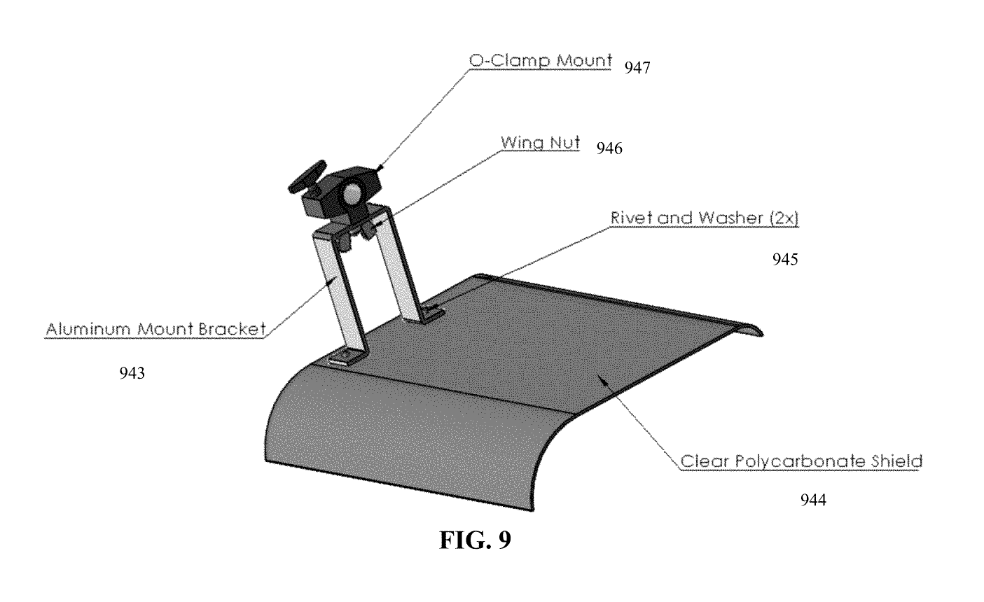

FIG. 9 illustrates mounting bracket 943 attached to shield 944. Mounting bracket 943 having 0-clamp 947 and wing nut 946.

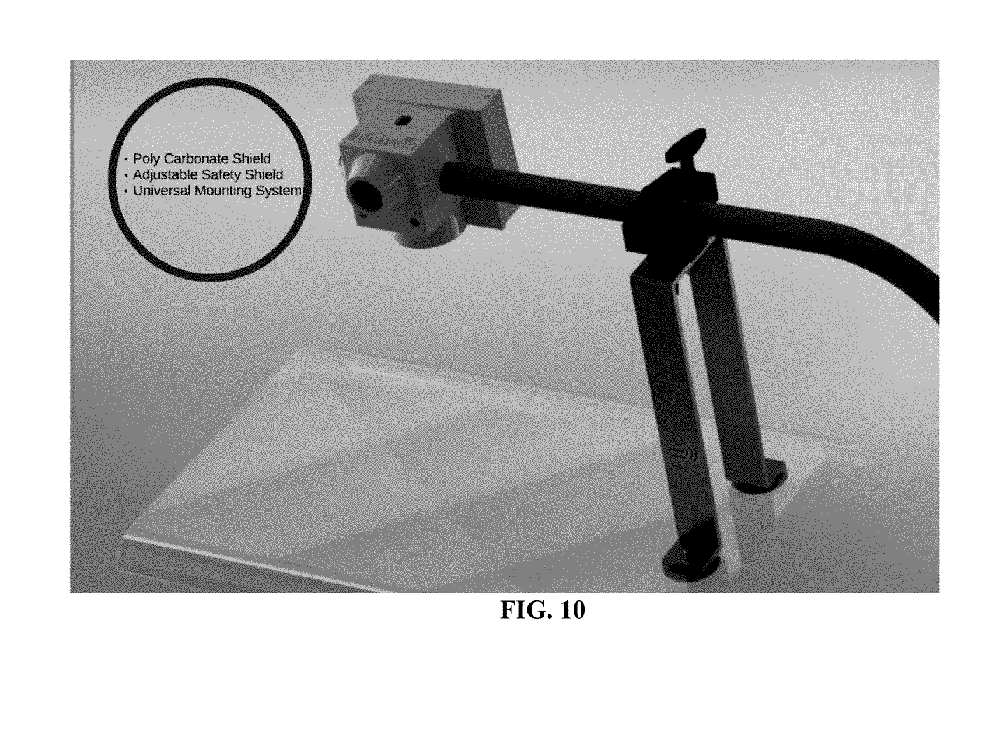

FIG. 10 illustrates a dual camera assembly attached to a shield via a mounting assembly of FIG. 9.

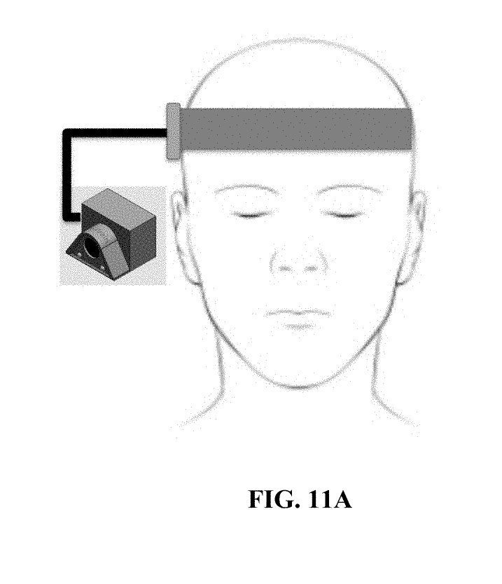

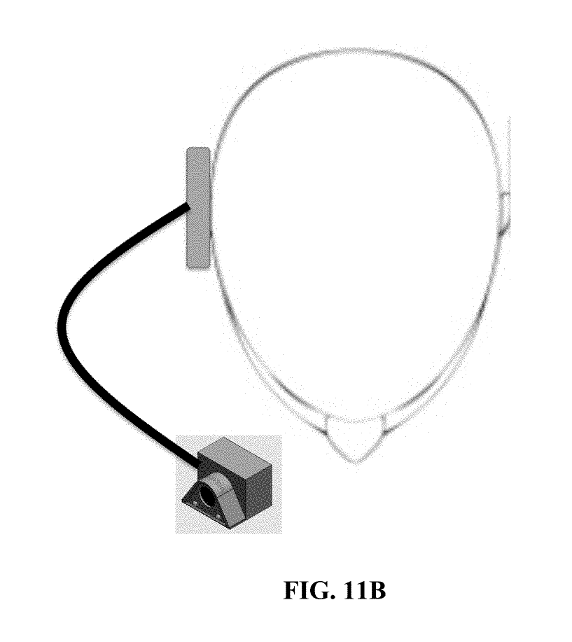

FIGS. 11A-11B. Illustration of head mountable assembly with flexible support. (A) frontal view. (B) top view.

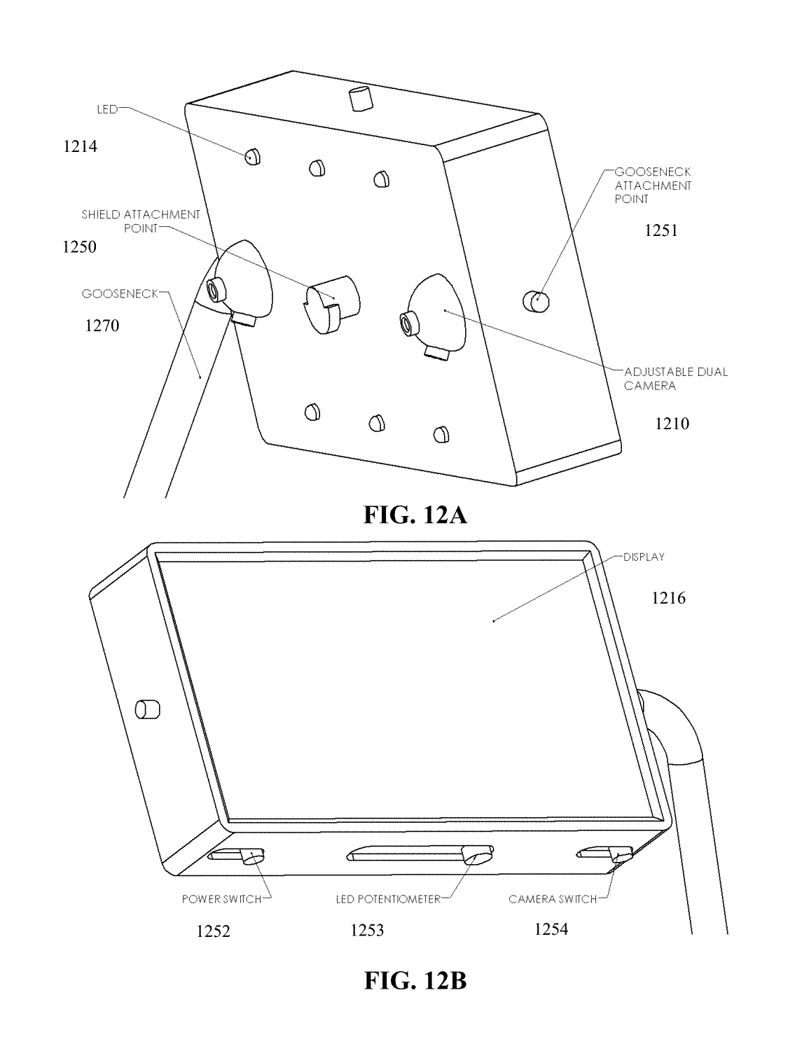

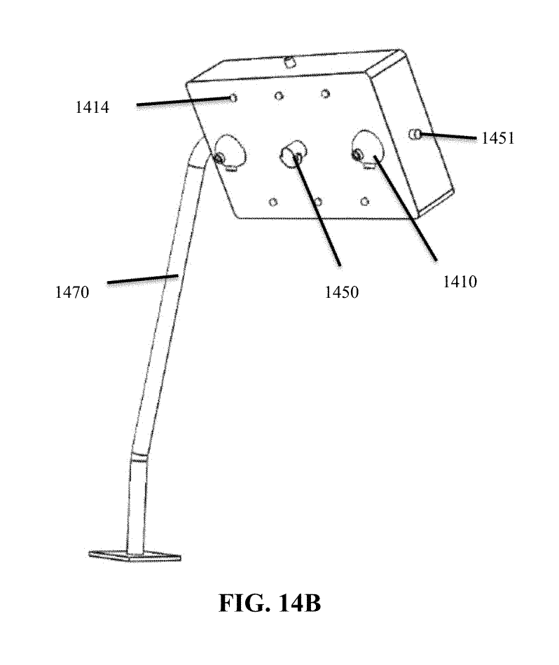

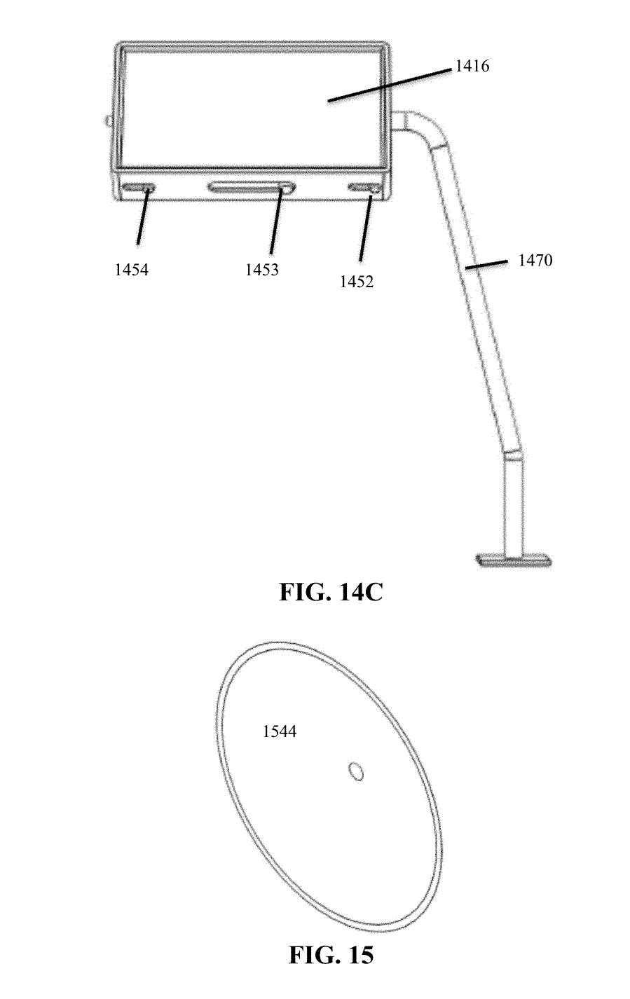

FIGS. 12A-12B and FIGS. 14A-14C illustrate one embodiment of a device that includes a camera assembly coupled to a gooseneck support.

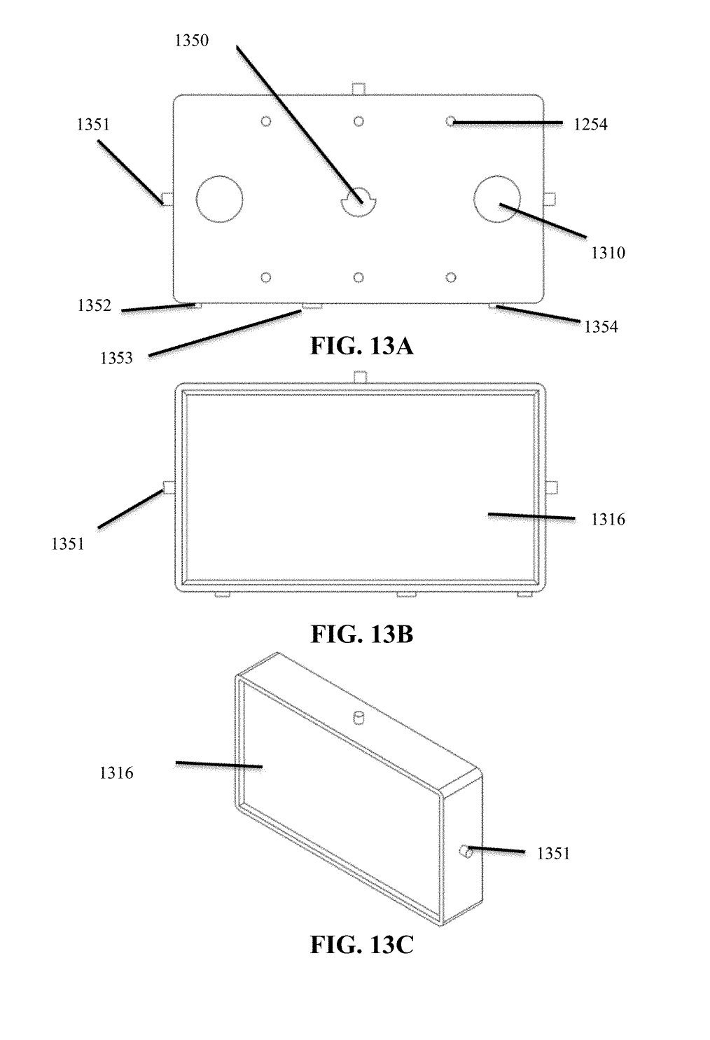

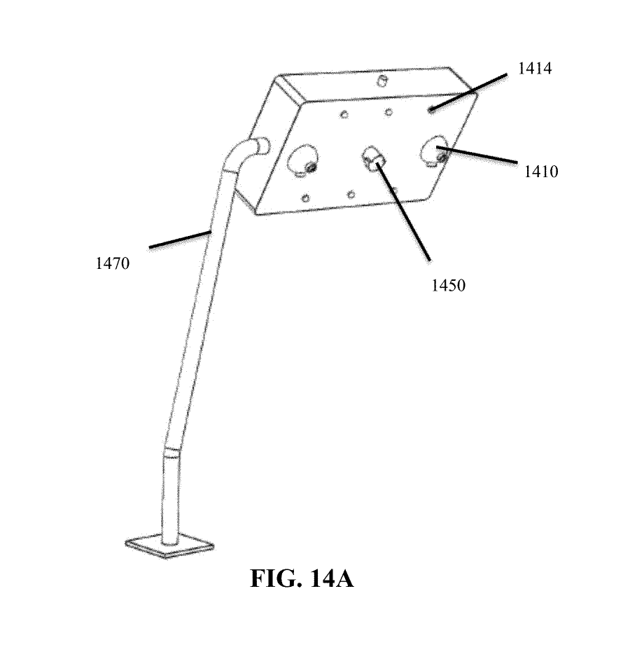

FIGS. 12A-12B, FIGS. 13A-13C, and FIGS. 14A-14B illustrate various embodiments of a dual camera assembly attached to a gooseneck support (1270, 1470). (A) frontal view with cameras. Adjustable dual camera 1210, 1310, 1410 is mounted in the assemble as are NIR LEDs 1214, 1314, 1414. The housing also includes gooseneck attachment points 1251, 1451 and shield attachment point 1250, 1350, 1450. (B) shows a back view with display 1216, 1316, 1416, power switch 1252, 1352, 1452, LED potentiometer 1253, 1353, 1453, and camera switch 1254, 1354, 1454.

In certain aspects a device includes one or more of a NIR camera assembly (e.g., FIGS. 5, 6, 12A-12B, and 13A-13B), a support that can be a flexible support (1214, 1414) (e.g., FIGS. 12A-12B, and 14A-14C), and a battery housing/attachment base (e.g., FIGS. 1, 7). In a further aspect the attachment mechanism is a screw (see item 8, FIG. 7) or magnetic attachment mechanism that is designed as a universal attachment that can be adapted to fit any number of attachment sites. The battery housing/attachment base is also configured to provide power to the NIR camera assembly (see battery 4 and power switch 3 of FIG. 7).

The batteries for the device can be charged through the use of wires between an electrical source and the batteries, wirelessly, such as through inductive charging, or by both. In some instances, the device includes a charging station. The charging station can be attached to the device or be separate from the device.

The camera assembly is coupled to the battery housing/attachment base by a support. The support can be a flexible support. The support can be a gooseneck support. The flexible support can be configured to move in a variety of directions yet maintain a position once the camera assembly is put in place by a user. In one example the battery housing/attachment base can be attached to a helmet and positioned on the side of a user's head. The flexible support can be manipulated to position the display of the camera assembly within view of the user when needed and moved out of the field of view when not needed. See, FIGS. 11A-11B, 12A-12B, and 14A-14C).

The camera assembly can include one or more camera(s). The camera(s) can be NIR camera(s). In some instances when multiple cameras are used, the use of the cameras can be switched or multiple cameras can be used as the same time. In some instances, the cameras are spatially adjustable to accommodate different viewing angles without having to adjust the camera assembly and/or the device. The camera assembly can have multiple attachments points for one or more support(s) to attach. The device can include a power switch to turn the power on or off to the device, camera assembly, and/or camera, etc. The device can include a camera selection switch to select use of one or more camera(s). The device can contain a potentiometer switch to regulate the intensity of a light associated with the device. The power switch, camera selection switch, and/or potentiometer switch can be on the camera assembly. The light can be one or more LED light(s). The device can have an attachment for a protective shield. The attachment for the protective shield can be on the camera assembly.

Near infrared vein imaging systems allow the operator to identify veins based on their absorbance behavior in the presence of near infrared (NIR) light. Deoxyhemoglobin in the veins absorbs near infrared light while surrounding tissues, e.g., fat, skin, etc., scatter and reflect near infrared light. The camera(s) in the device can be NIR cameras. The cameras can be auto adjusting cameras. The cameras can auto adjust based on the environment. The cameras can be associated with, include, or be compatible with imaging software. The cameras can be associated with, include, or be compatible with imaging filters, such as electromagnetic wavelength filters. The cameras or imaging software can be capable of filtering out different electromagnetic wavelengths and/or different ambient light. In some instances, the camera(s) are capable of capturing one or more electromagnetic wavelengths from 700-1000 nm. In some instances, the camera(s) can be capable of recording still images or video.

The device can include a user display. The user display can display the image and/or video captured by the camera(s) and/or stored image(s) or video(s). The display can be included on the camera assembly or be separate from the device or camera assembly. The display can be connected by wire or wirelessly to the camera(s). The display can be configured to control the camera(s), such as by enabling a user to activate auto adjust, to focus, to use one or more camera(s), to use one or more imaging filters, etc. The display can also be used to display a user interface for imaging software. In some instances, the display is a touch screen.

The device can be associated with, include, or be compatible with videogrammetry imaging software. Videogrammetry software can determine the three-dimensional coordinates of an object or points on an object. Two or more cameras on the device can be used to determine the three-dimensional coordinates. In some instances, videogrammetry is used to provide three-dimensional depth perception for venipuncture procedures.

The device can be associated with, include, or be compatible with digital memory. The digital memory can be internal memory or external to the device. The memory can be cloud based memory. The memory can be accessed through wiring or wirelessly, such as through Bluetooth. The memory can be capable of recording video from the camera(s), store videos, store a graphical user interface, store software, store software updates, store software patches, etc., or a combination thereof.

The device can be capable of receiving, using, and/or displaying live video, stored video, an image, a graphical user interface(s), software, software updates, software patches, etc., or a combination thereof. The device can use wired or wireless, such as Bluetooth, connections to enable such and/or to connect to components of the device and/or to another device, network, internet, the cloud, etc.

The device can be associated with, include, or be compatible with a software application (“app”). The app can be stored, controlled, or accessed by a personal device, such as a smart phone, computer, or device with a display. The app may be a cloud based app. The app may be capable of receiving, using, and/or displaying video. The app may be capable of receiving, using, and/or displaying diagnostics on the device. The device can connect to the app. The device can connect to the app through wired or wireless, such as Bluetooth, connections.

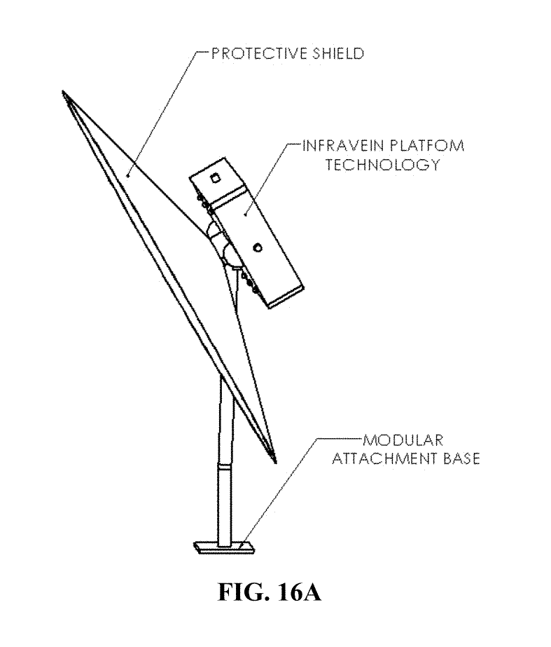

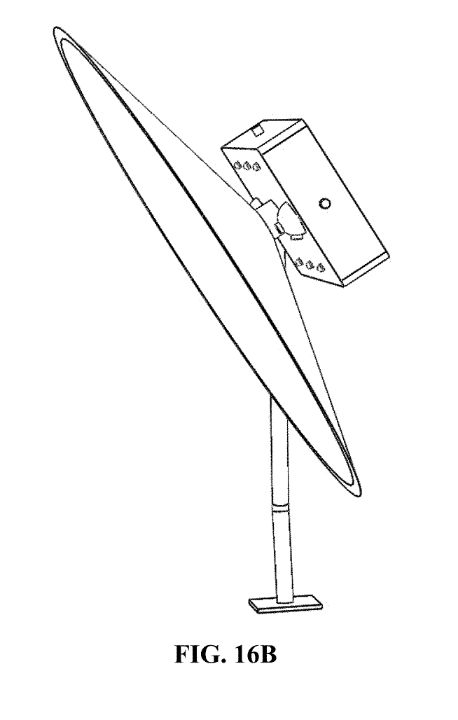

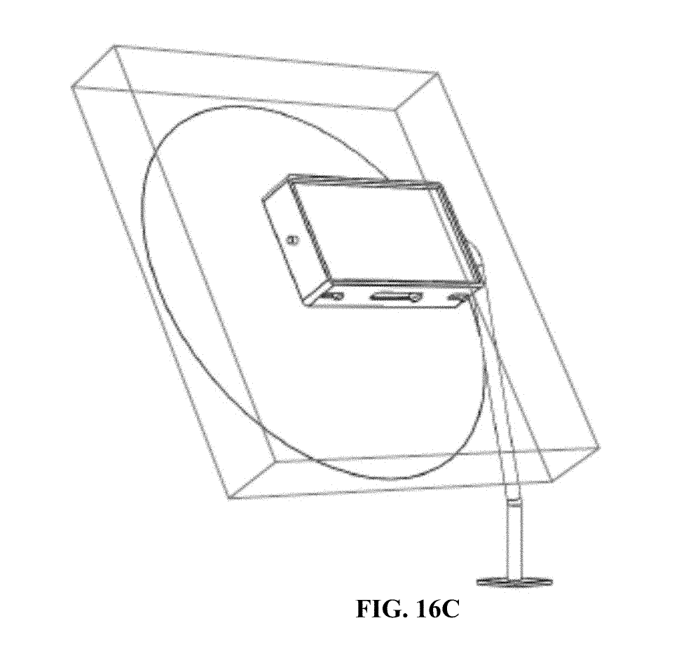

In other aspects the device can be configured for user protection by attaching a blood spatter shield, thereby protecting the clinician and surrounding personnel from exposure to potentially infected blood during venipuncture. In some instances, the splatter shield can be attached to the camera assembly or the support for the camera assembly, such as a gooseneck support. In some instances, the height of the splatter shield is adjustable. The splatter shield can be easily detached. The splatter shield can be easily cleaned and sterilized without compromising transparency. The splatter shield can be designed to meet Occupational Safety and Health Administration (OSHA) requirements for personal protective equipment for users who handle blood. In certain aspects the splatter shield is transparent to NIR and/or light in the visible spectrum. In a further aspect the shield is a polycarbonate shield. See, FIGS. 9, 10, 15, and 16A-16C.

The device adds portability by providing the user the ability to mount the device to a variety of surfaces in many environments, including mobile ambulances, trauma centers, mobile and non-mobile blood donation stations, and headgear or harness worn by the user. This can be realized by use of a modular, central unit, and environment-specific mounts (e.g., FIGS. 11, 14A-14C). The mount can include one or more variable attachments. The variable attachments can be a spring loaded connected c-clamp, curved c-clamp, magnetic clamp, helmet and/or harness attachment, magnetic surface, or a rubber surface. The spring loaded connected c-claim can have flat clamping edges that is capable of closing and opening to at least 15 cm. The curved c-clamp can be an adjustable c-curved clamp that is capable of closing and opening to at least 15 cm, and may depending on the thickness of the intended surface site. The magnetic clamp can include a magnetic strip on the surface of the clamp. The clamps can be clamped to any flat edged surface and in some instances, can have extended flaps that will grip onto surfaces for leverage. The helmet and/or harness attachment can be capable of attaching to a helmet, a harness, or both. The magnetic surface can be included on any of the attachments or may be a separate magnetic surface. The rubber surface can increase the friction between the device and the intended surface. The rubber surface can be included on any of the attachments or may be a separate rubber surface. The rubber surface can be a high friction rubber. The rubber surface can be rubber on the exterior with a putty inner core. Any of the mounts can include a pivot to attach a support, such as a gooseneck or a flexible support.

In addition to the clinical practice applications, the devices described herein can be used for clinical training. The device would be utilized in conjunction with existing phlebotomy training techniques to hasten venipuncture proficiency in both normal and complicated conditions.

1. A vein visualization device comprising:
(i) a near infrared camera assembly;
(ii) a battery/attachment base; and
(iii) a flexible support connecting the camera assembly and the battery/attachment base.

2. The device of claim 1, wherein the battery/attachment base comprises a screw type attachment mechanism.

3. The device of claim 1, wherein the camera assembly comprises two cameras.

4. The device of claim 3, wherein the two cameras are configured for videogrammetry.

5. The device of claim 1, further comprising a splatter shield.

6. The device of claim 1, further comprising a head gear attached to the battery/attachment base, where the flexible support is configured to allow the camera assembly to be position within the visual field of a user.
